(19) United States
(12) Patent Application Publication
(10) Pub. No.: US 2015/0174569 A1
(43) Pub. Date: Jun. 25, 2015

(54) PHOTOCATALYTIC TIO2 COATINGS ON THE POLYMER SURFACES ACTIVATED WITH VISIBLE LIGHT, METHOD OF THEIR PREPARATION AND USE THEREOF

(71) Applicant: UNIWERSYTET JAGIELLONSKI, Kraków (PL)

(72) Inventors: Wojciech Macyk, Liszki (PL); Rafal Sadowski, Krakow (PL); Przemyslaw Labuz, Krakow (PL); Marta Buchalska, Krakow (PL)

(73) Assignee: Uniwersytet Jagiellonski, Kraków (PL)

(21) Appl. No.: 14/416,097

(22) PCT Filed: Jul. 22, 2013
(86) PCT No.: PCT/EP2013/065400

§ 371 Date: Jan. 21, 2015

(30) Foreign Application Priority Data
Jul. 23, 2012 (PL) PL400098

(51) Int. Cl. B01J 31/38; B01J 35/00; A61L 2/08; B01J 37/34

(52) U.S. Cl. CPC B01J 31/38; B01J 37/349; B01J 35/0006; B01J 35/004; A61L 2/088; B01J 2231/005; B01J 2531/002; B01J 2531/008

(57) ABSTRACT

The invention relates to visible-light active photocatalytic coatings of titanium oxide (IV) on polymer surfaces, characterized in that are applied to a substrate which is the organic polymer material used in medicine, food and pharmaceutical packaging, and the coating is nanocrystalline titanium oxide (IV) or nanocrystalline titanium oxide (IV) surface-modified with organic compound. The invention also includes a method for the preparation of photocatalytic coatings of titanium oxide (IV) according to the invention, comprising the step of activating the surface of a polymeric material with low-temperature plasma technology, and then the synthesis of nanocrystalline titanium oxide (IV) coating and the modification of the surface layer with organic compounds. The invention also relates to the application of photocatalytic coating of titanium oxide (IV) according to the invention for sterilization of plastic elements used in medicine, food and pharmaceuticals packaging and application to production of selected products from the group consisting of: photosterilizing materials, photobactericidal, photofungicidal, photocatalytic materials.

### TECHNICAL FIELD

The subject of the invention are photocatalytic titanium oxide(IV) coatings on the polymer surfaces activated with visible light, method for their preparation and their use. The polymer surfaces are selected from the group of polymers used in medical and food packaging, in particular polyurethane (PU), polycarbonate (PC), polyvinyl chloride (PVC), polytetrafluoroethylene (PTFE).

### STATE OF ART

There has been a significant increase in the use of plastics in almost every field of human activity in recent years. The low price and simplicity of adaptation of materials to the requirements of a developing society is contributed to this particularly. Polymeric materials also play an increasingly important role in medicine, since the disposable medical equipment is made of these materials. The surface of the plastics is exposed to the formation of biofilm what is dangerous to health and what is one of the most important etiological contribution to the nosocomial infections. According to World Health Organization, the minimization of the nosocomial infections positively impacts on the patients' health as well as on the economic condition of medical facilities. The answer to the problem of nosocomial infections is to design new materials with potential antibacterial effect. The increase in resistance of pathogens to conventionally available antimicrobial agents requires exploration of other innovative solutions.

The answer to the problem is utilization of photocatalytic materials, particularly semiconductors, for example titanium oxide(IV), in photoinactivation of the bacteria. The present state of art discloses such applications, however, titanium oxide(IV) usually occurs in the form of powders, solutions or colloidal layers at mineral surfaces such as glass or metal (Chen, X., Mao, S. S, 2007, Chemical Reviews, 107, 2891-2959), what significantly reduces the possibility of its application.

The activation process of the polymer surfaces by low-temperature plasma technique is well known and described in the literature by Hegemann (D. Hegemann et al., Nuclear Instruments and Methods in Physics Research B 2003, 208, 281-286), Kasanen (J. Kasanen et al., 2009, J. Appl. Polym. Sci., 111, 2597-2606) and Vandencasteele (N. Vandencasteele et al., 2010, Journal of Electron Spectroscopy and Related Phenomena, 178-179, 394-408). The activation of the polymer surfaces changes their physical and chemical properties. As a result of the activation process, many oxygen-containing functional groups are generated at the surface of polymeric material in the presence of oxygen, such as hydroxyl group (—OH), carboxyl group (—COOH), carbonyl group (—C═O).

The methods of modifying of the material properties without the changes in volume by applying plasma treatment are also known. For instance the plasma treatment used for modifying the optical fiber cable is disclosed in patent application GB 2481891 A. The entire length of optical fiber is subjected to plasma treatment by moving it through the plasma furnace, thereupon the surface of plasma-treated optical fiber is modified becoming more susceptible to combining it with the target substance. The method allows for creation a layer of target substance adhering to the surface of optical fiber cable. The materials used in abovementioned example for the adhering layer formation are epoxy resins, polyamide and polyurethane. Inconvenience observed in the prior art relates to the difficulty in obtaining sustained coatings of titanium oxide(IV) on the polymer surfaces and their sensitization to the visible light range. Permanent immobilization of titanium oxide(IV) on the polymer surfaces provides new opportunities for the application of photocatalytic processes at TiO₂, for example to photosterilize of various medical materials made of plastics.

The aim of the present invention is to provide sustained photocatalytic coatings of titanium oxide(IV) on the polymer surfaces, activated with visible or ultraviolet light, exhibiting high photocatalytic and photosterilizing activity and the method of their preparation. The present invention provides also new opportunities for the application of coatings according to the invention, a certain set of characteristics to the sterilization of various materials, such as medical catheters or food packaging.

### DETAILED DESCRIPTION OF THE INVENTION

The invention relates to the photocatalytic coatings of titanium oxide(IV) on the polymer surfaces activated with visible light, characterized by:

a) their application on organic substrate which is a polymeric material used in medicine, food and pharmaceuticals packaging, in particular selected from the group of: polyurethane (PU), polycarbonate (PC), polyvinyl chloride (PVC), polytetrafluoroethylene (PTFE).
b) coating of nanocrystalline titanium oxide(IV) or nanocrystalline titanium oxide(IV) surface-modified with organic compounds.

Preferably, nanocrystalline titanium oxide(IV) is surface-modified with organic compound selected from the group consisting of:

a) compound with Formula 1:

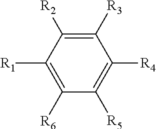

where: R₁-R₄ are —H, saturated or unsaturated substituents, —NH₂, —NH₃⁺ or —SO₃M, wherein M is: H⁺, K⁺, Na⁺, Li⁺, NH₄⁺, and R₅ i R₆ are —OH or —COOH,
   b) ascorbic acid,
   c) compound with Formula 2 (rutoside):

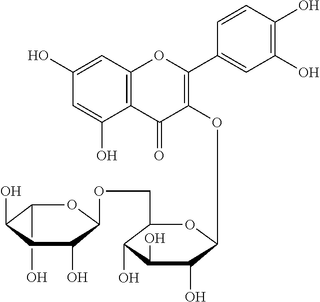

Preferably, organic compound is selected from the group consisting of: phthalic acid, 4-sulfophthalic acid, 4-amino-2-hydroxybenzoic acid, 3-hydroxy-2-naphthyl acid, salicylic acid, 6-hydroxysalicylic acid, 5-hydroxysalicylic acid, 5-sulfosalicylic acid, 3,5-dinitrosalicylic acid, 1,4-dihydroxy-1,3-benzenedisulfonic disodium salt, gallic acid, pyrogallol, 2,3-naphthalenediol, 4-methylcatechol, 3,5-di-tert-butylcatechol, p-nitrocatechol, 3,4-dihydroxy-L-phenylalanine (DOPA), catechol, rutoside and ascorbic acid.

The invention relates also to the method of preparation of titanium oxide(IV) photocatalytic coatings activated with visible light on the surfaces of the polymers used in medicine, food and pharmaceuticals packaging, in particular selected from the group of: polyurethane (PU), polycarbonate (PC), polyvinyl chloride (PVC), polytetrafluoroethylene (PTFE) and is characterized in that it comprises three stages:

a) surface activation of a polymer material with low temperature plasma technique,
b) synthesis of nanocrystalline coating of titanium oxide(IV) using a suspension of nanocrystalline titanium oxide(IV),
c) modification of the coating surface using organic compounds by means of application of the modifier in solution.

Preferably, the activation takes place under the influence of the oxygen or nitrogen plasma. Preferably, the activation takes place at a plasma pressure of 0.1-1 mbar with the time of plasma operation between 5-500 s. Also preferably, when polymeric materials activated with plasma are left in the air to form oxygen-containing functional groups.

Also preferably, if nanocrystalline titanium oxide(IV) with a grain size up to 100 nm in the form of an aqueous colloidal solution is used for the synthesis. Preferably, the synthesis process is carried out by the immersion techniques (for instance dip-coating technique), spraying or painting techniques. Preferably, the process takes place at room temperature.

Preferably, surface modification of coating with organic compounds takes place in aqueous or alcoholic solution of modifier with minimum concentration 10⁻⁴ mol/dm³ followed by drying.

Preferably, organic compound is selected from the group consisting of: phthalic acid, 4-sulfophthalic acid, 4-amino-2-hydroxybenzoic acid, 3-hydroxy-2-naphthyl acid, salicylic acid, 6-hydroxysalicylic acid, 5-hydroxysalicylic acid, 5-sulfosalicylic acid, 3,5-dinitrosalicylic acid (Table 1), 1,4-dihydroxy-1,3-benzenedisulfonic disodium salt, gallic acid, pyrogallol, 2,3-naphthalenediol, 4-methylcatechol, 3,5-di-tert-butylcatechol, p-nitrocatechol, 3,4-dihydroxy-L-phenylalanine (DOPA), catechol (Table 2), rutoside and ascorbic acid.

TABLE 1
Phthalic acid and salicylic acid derivatives.

| Compound | | |
|---|---|---|
| symbol | Compound name | Structural formula |
| A-1 | phthalic acid | 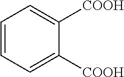 [SMILES: O=C(O)c1ccccc1C(=O)O] |
| A-2 | 4-sulfophthalic acid | 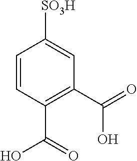 [SMILES: O=C(O)c1ccc(S(=O)(=O)O)cc1C(=O)O] |
| S-1 | 4-amino-2-hydroxybenzoic acid | 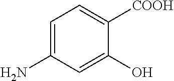 [SMILES: Nc1ccc(C(=O)O)c(O)c1] |
| S-2 | 3-hydroxy-2-naphthyl acid | 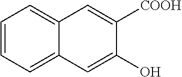 [SMILES: O=C(O)c1cc2ccccc2cc1O] |
| S-3 | salicylic acid | 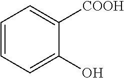 |
| S-4 | 6-hydroxysalicylic acid | 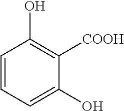 [SMILES: O=C(O)c1c(O)cccc1O] |
| S-5 | 5-hydroxysalicylic acid | 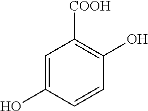 [SMILES: O=C(O)c1cc(O)ccc1O] |
| S-6 | 5-sulfosalicylic acid | 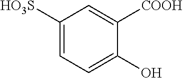 [SMILES: O=C(O)c1cc(S(=O)(=O)O)ccc1O] |
| S-7 | 3,5-dinitrosalicylic acid | 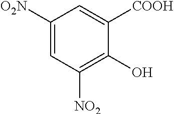 [SMILES: O=C(O)c1cc([N+](=O)[O-])cc([N+](=O)[O-])c1O] |

TABLE 2
Catechol derivatives.

| Compound | | |
|---|---|---|
| symbol | Compound name | Structural formula |
| K-1 | 1,4-dihydroxy-1,3- benzenedisulfonic disodium salt | 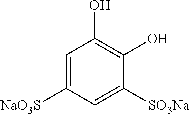 |
| K-2 | gallic acid | 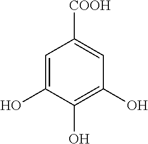 [SMILES: O=C(O)c1cc(O)c(O)c(O)c1] |
| K-3 | pyrogallol | 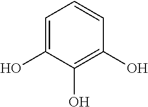 |
| K-4 | 2,3-naphthalenediol | 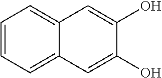 [SMILES: Oc1cc2ccccc2cc1O] |
| K-5 | 4-methylcatechol | 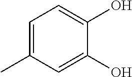 |
| K-6 | 3,5-di-tert-butylcatechol | 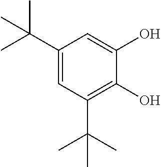 [SMILES: CC(C)(C)c1cc(O)c(O)c(C(C)(C)C)c1] |
| K-7 | p-nitrocatechol | 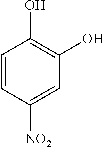 [SMILES: O=[N+]([O-])c1ccc(O)c(O)c1] |
| K-8 | 3,4-dihydroxy-L- phenylalanine (DOPA) | 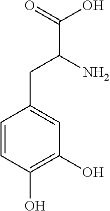 [SMILES: NC(Cc1ccc(O)c(O)c1)C(=O)O] |
| K-9 | 1,2-dihydroxy-benzene (catechol) | 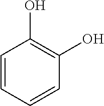 |

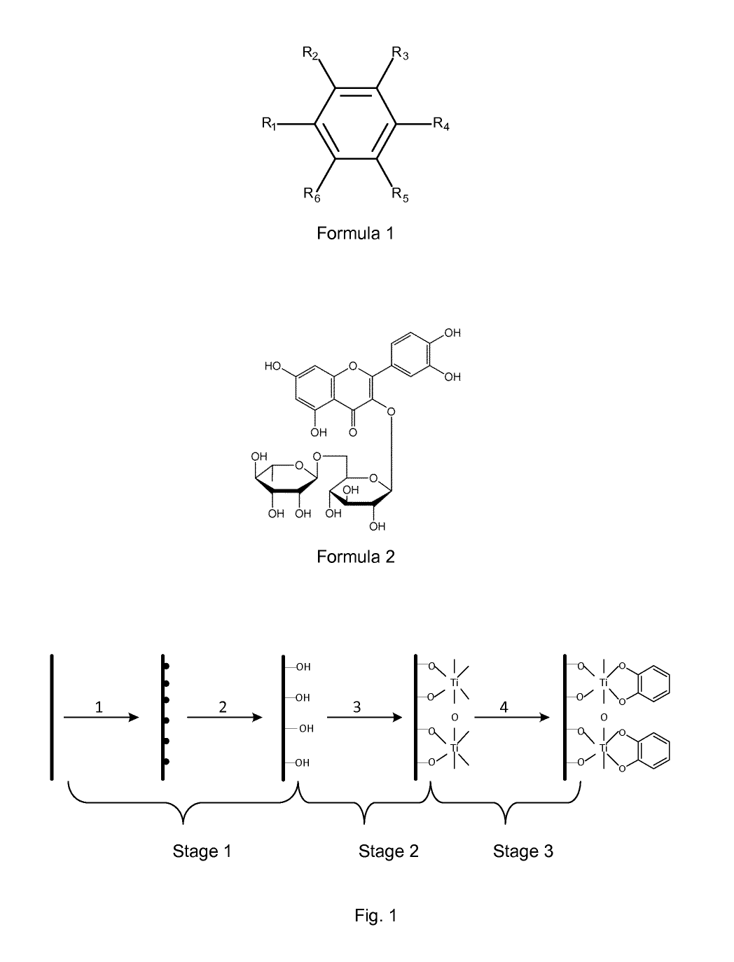

Particular steps of the method are also graphically illustrated in FIG. 1.

Another subject of the invention is the use of the photocatalytic coatings of titanium oxide(IV), as defined above activated with visible light on the surfaces of polymers, and/or obtainable in the method according to the invention, for sterilization of plastic components used in medicine, food and pharmaceuticals packaging, in particular selected from the group of: polyurethane (PU), polycarbonate (PC), polyvinyl chloride (PVC), polytetrafluoroethylene (PTFE).

Particular subject of the invention is use of the coatings according to the invention and/or obtained by the process according to the invention for the production of selected products from the group consisting of: photosterilization materials, photobactericidal, photofungicidal, photocatalytic and other materials, especially transparent, for instance medical catheters, medical plastic tubes, foil and packaging for food and pharmaceuticals.

Particular subject of the invention is use of the coatings according to the invention and/or obtained by the process according to the invention, in medicine (for example in dermatology, ophthalmology, otolaryngology, urology, gynecology, rheumatology, oncology, surgery, hospitality, veterinary medicine and dentistry) and in cosmetology, preferably, for the sterilization of medical catheters, plastic tubes and other surfaces, sterilization which is advantageous and/or required.

### SUMMARY OF THE INVENTION

The material according to the present invention exhibits photocatalytic activity when irradiated with visible light (l>400 nm; photocatalysis is a consequence of the absorption of visible light by surface complex of titanium, type charge-transfer) and ultraviolet light (l<400 nm; photocatalysis is a consequence of the absorption of ultraviolet light by surface complex of titanium, type charge-transfer or directly by titanium oxide(IV)). As a result of light exposure, Reactive Oxygen Species are formed (OH., O₂⁻, H₂O₂, ¹O₂), which are responsible for oxidation of organic compounds and decay of microorganisms.

The present invention in embodiments not limiting the scope of its application is presented in FIGS. 1-6, in which:

FIG. 1 shows a general diagram of a method for preparing the photocatalytic coating of titanium oxide(IV) activated with visible light, on polymer surfaces,

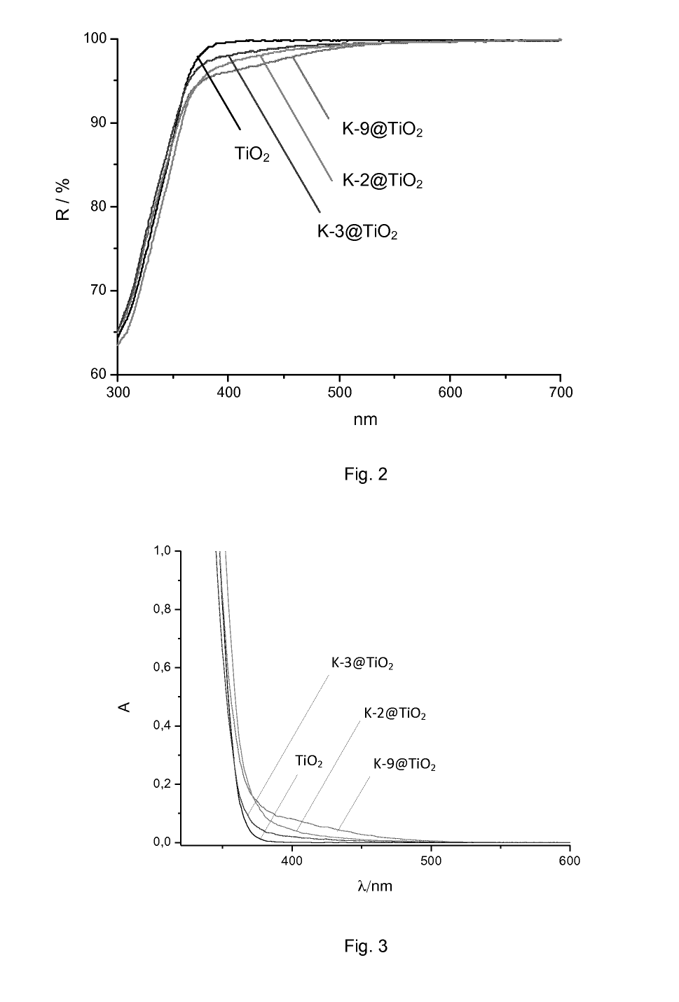

FIG. 2 shows UV-vis diffuse-reflectance spectrum of PTFE coated with TiO₂ and modified with various modifiers,

FIG. 3 shows the transformation of UV-vis diffuse-reflectance spectrum into UV-vis absorbance spectrum of PTFE coated with TiO₂ and modified with various modifiers,

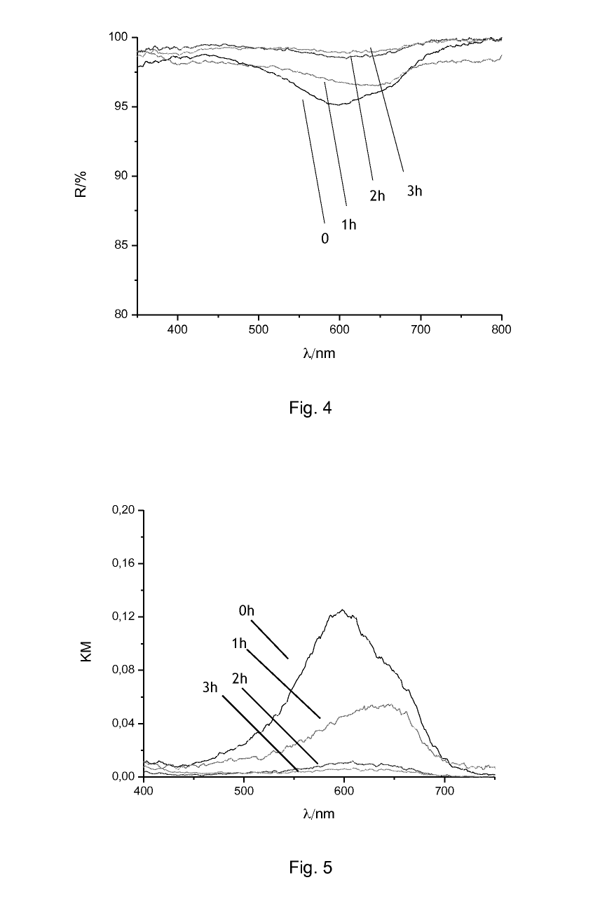

FIG. 4 shows photodegradation of azure B “dry mode”, diffuse-reflectance spectra of TiO₂@PTFE with adsorbed model pollution (azure B), irradiation: XBO-150, water filter, l>320 nm,

FIG. 5 shows photodegradation of azure B “dry mode”, diffuse reflectance spectra of TiO₂@PTFE with adsorbed model pollution (azure B) transformed by Kubleka-Munk function, irradiation: XBO-150, water filter, l>320 nm,

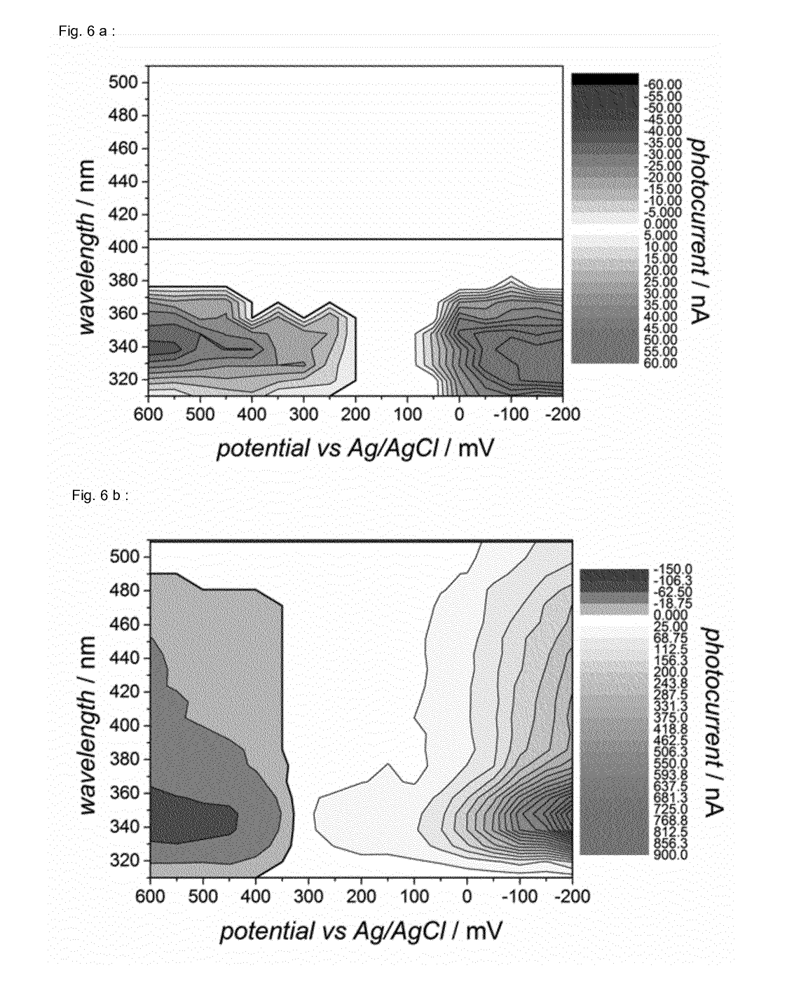

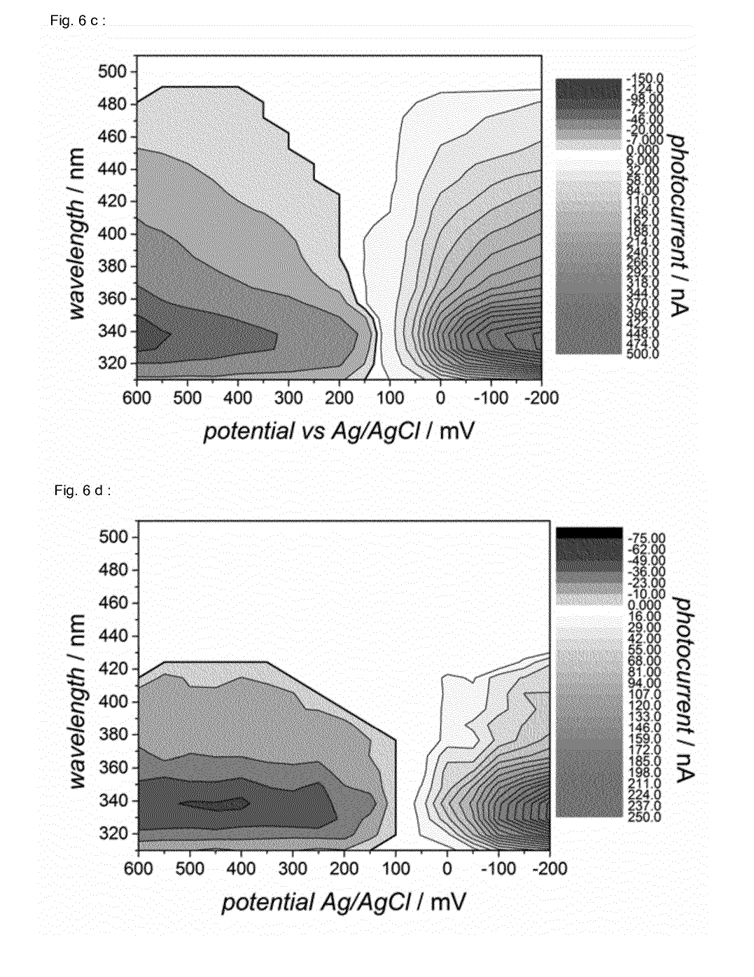

FIG. 6 shows the surface modification with catechol and salicylic acid, which results in well-defined extension of the scope of activity of the layers to visible light K-9@TiO₂ (b), S-2@TiO₂ (c), K-4@TiO₂ (d) in comparison to the layer made of unmodified TiO₂ (a).

### EXAMPLE 1

### Obtaining Visible-Light Active, Nanocrystalline Photocatalysts as Coatings on Polymer Surfaces

Starting substrates for the synthesis of described materials are:

a) commercially available plastic materials, particularly polyurethane (PU), polycarbonate (PC), polyvinyl chloride (PVC), polytetrafluoroethylene (PTFE),
b) unmodified, nanocrystalline titanium oxide(IV) in the form of a colloidal, aqueous solution having a particle size less than 100 nm and organic surface modifiers.

### Variant 1

A square with a side length of 3 cm was cut from a commercially available film of polytetrafluoroethylene (PTFE) and purified with detergent. The surface was activated with oxygen plasma in conditions: p=0.3 mbar, t=120 s, v=2 cm³/min, power 100 W. After activation process, the polymer material was left in the air for 2 minutes. The synthesis of the coating was carried out using immersion technique (dip-coating) from colloidal solution of nanocrystalline titanium oxide(IV) (15% wt). The drawing speed of polymer material from the solution was established at 1 cm/min at room temperature. As a result, uniform, durable coating of TiO₂ was created at the surface of foil. The resulting coatings were modified using impregnation by immersing the samples for 2 min in a solution of the modifier from the group S (S-2, S-3; Table 1) or group K (K-1, K-4, K-9; Table 2) or ascorbic acid or rutoside in the form of aqueous solution with concentration 10⁻⁴ mol/dm³ and then dried in the air.

### Variant 2

A square with a side length of 3 cm was cut from a commercially available film of polyurethane (PU) and purified with detergent. The surface was activated with oxygen plasma in conditions: p=0.4 mbar, t=60 s, v=2 cm³/min, power 100 W. After activation process, the polymer material was left in the air for 2 minutes. The synthesis of the coating was carried out using immersion technique (dip-coating) from colloidal solution of nanocrystalline titanium oxide(IV) (4% wt). The drawing speed of polymer material from the solution was established at 1 cm/min at room temperature. As a result, uniform, durable coating of TiO₂ was created at the surface of foil. The resulting coatings were modified using impregnation by immersing the samples for 2 min in a solution of the modifier from the group S (S-2, S-3; Table 1) or group K (K-1, K-4, K-9; Table 2) or ascorbic acid or rutoside in the form of aqueous solution with concentration 10⁻⁴ mol/dm³ and then dried in the air.

### Variant 3

A square with a side length of 3 cm was cut from a commercially available film of polycarbonate (PC) and purified with detergent. The surface was activated with oxygen plasma in conditions: p=0.4 mbar, t=120 s, v=2 cm³/min, power 100 W. After activation process, the polymer material was left in the air for 2 minutes. The synthesis of the coating was carried out using immersion technique (dip-coating) from colloidal solution of nanocrystalline titanium oxide(IV) (15% wt). The drawing speed of polymer material from the solution was established at 1 cm/min at room temperature. As a result, uniform, durable coating of TiO₂ was created at the surface of foil. Prepared coatings were modified using impregnation by immersing the samples for 2 min in a solution of the modifier from the group S (S-2, S-3; Table 1) or group K (K-1, K-4, K-9; Table 2) or ascorbic acid or rutoside in the form of aqueous solution with concentration 10⁻⁴ mol/dm³ and then dried in the air.

### EXAMPLE 2

### Characterization of Obtained Materials

Coatings synthesized according to the Example 1 are permanently affixed to the substrate and indelible. UV-vis spectra of the coatings modified with compounds from the group K are presented in FIG. 2 and FIG. 3. There is a distinct absorption of visible light, preferably in the wavelength range around 500 nm.

The activity of obtained coatings was specified using “dry mode”. Aqueous solutions of azure B were used as model pollution for photocatalytic activity tests. A sample of plastic with the synthesized TiO₂ coating was impregnated in a solution of pigment with a concentration 2.5·10⁻⁴ mol/dm³ for 1 h. Afterwards, the sample was irradiated for 3 h and the changes occurring during the experiment were monitored. The measuring system consisted of the xenon lamp XBO-150. Directly behind the lamp a water filter with copper sulfate(II) solution was placed (which cuts off radiation from the near infrared range, l>700 nm) and a high-pass filter which transmits radiation in the range l>320 nm. The sample was placed at a distance of 40 cm from the light source. Changes in the intensity of the dye colour were assessed during irradiation by spectrophotometry. The diffuse-reflectance spectrum and absorbance spectrum of azure B degradation during irradiation are shown in FIG. 4 and FIG. 5. Almost complete degradation of the dye at the time of 3 h was observed.

### EXAMPLE 3

### Studies on Photoactivity of Obtained Coatings

Coatings synthesized according to the Example 1 were tested to determine the spectral range of their activity. For photoelectrochemical measurements the set which included: xenon lamp XBO-150 (Osram) with power 150 W and power supply adaptor LPS-250 (Photon Technology International), monochromator with the shutter and electrochemical analyzer BAS-50W (Bioanalytical systems), was used. The control system of shutter and monochromator allows automatic control of wavelength of the incident light and the time of exposure to radiation of the working electrode. Photoelectrochemical measurements were carried out in three-electrode cell, in quartz cuvette with volume 25 cm³ filled with electrolyte (KNO₃ solution with concentration 0.1 mol dm⁻³). A platinum electrode was used as an auxiliary electrode and silver chloride electrode (Ag/AgCl)—as a reference electrode. Working electrode was the ITO film (transparent electrically conductive material, a mixture of indium-tin oxide) coated with a layer of nanocrystalline TiO₂ modified with organic compounds according to the method described in Example 1. The idea of the photoelectrochemical experiment is to measure the intensity of generated photocurrent as a function of light wavelength (l=f(I)) incident at photoelectrode in the form of short flashes (10 s.), at constant potential applied to the working electrode. The coatings were irradiated with monochromatic light in the wavelength range of 330-700 nm, changing with each flash of fixed interval (9.5 nm). The change in wavelength occurred when the shutter was closed. The plots I=f(I) (share spectra without taking into account the absolute intensity of light) recorded in the potential range 600÷−200 mV were used to determine the photoreply profile as a function of wavelength and potential applied to the working electrode (so called photocurrent maps, l=f(E,l)). Surface modification with catechol derivatives and salicylic acid results in well-defined extension of the scope of activity to visible light (FIG. 6: K-9@TiO₂ (b), S-2@TiO₂ (c), K-4@TiO₂ (d)) in comparison to the coating made of unmodified TiO₂ (FIG. 6a).

1. An article of manufacture comprising
a) a substrate which is an organic polymer material used in medicine, food or pharmaceutical packaging, selected from the group consisting of polyurethane (PU), polycarbonate (PC), polyvinyl chloride (PVC), and polytetrafluoroethylene (PTFE), and
b) a coating on the substrate which coating nanocrystalline titanium oxide(IV), or nanocrystalline titanium oxide(IV) surface-modified with an organic compound.

2. The article according to claim 1, characterized in that the nanocrystalline titanium oxide(IV) is surface-modified with an organic compound selected from the group consisting of:
a) compound with Formula 1: 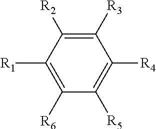
where: R₁-R₄ are —H, saturated or unsaturated substituents, —NH₂, —NH₃⁺ or —SO₃M, wherein M is: H⁺, K⁺, Na⁺, Li⁺, NH₄⁺, and R₅ i R₆ are —OH or —COOH,
b) ascorbic acid, and
c) compound with Formula 2 (rutoside):

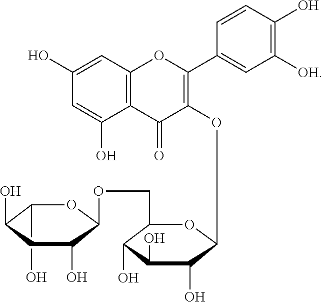

3. The article according to claim 1, characterized in that the nanocrystalline titanium oxide(IV) is surface-modified with an organic compound selected from the group consisting of: phthalic acid, 4-sulfophthalic acid, 4-amino-2-hydroxybenzoic acid, 3-hydroxy-2-naphthyl acid, salicylic acid, 6-hydroxysalicylic acid, 5-hydroxysalicylic acid, 5-sulfosalicylic acid, 3,5-dinitrosalicylic acid, 1,4-dihydroxy-1,3-benzenedisulfonic disodium salt, gallic acid, pyrogallol, 2,3-naphthalenediol, 4-methylcatechol, 3,5-di-tert-butylcatechol, p-nitrocatechol, 3,4-dihydroxy-L-phenylalanine (DOPA), catechol, rutoside and ascorbic acid.

4. titanium oxide(IV) photocatalytic coatings activated with visible light on the surfaces of polymers used in medicine, food and pharmaceuticals packaging, selected from the group consisting of: polyurethane (PU), polycarbonate (PC), polyvinyl chloride (PVC), polytetrafluoroethylene (PTFE), comprising
a) surface activation of a polymer material with low temperature plasma technology,
b) synthesis of nanocrystalline coating of titanium oxide(IV) using a suspension of nanocrystalline titanium oxide(IV),
c) modification of the coating surface using an organic compound by means of application of the modifier in solution.

5. The method according to claim 4, characterized in that the surface activation takes place under the influence of the oxygen or nitrogen plasma.

6. The method according to claim 4, characterized in that the surface activation takes place under plasma pressure 0.1-1 mbar and in time of plasma operation 5-500 s.

7. The method according to claim 4, characterized in that the polymeric material activated by plasma technology is left in the air to form oxygen-containing functional groups.

8. The method according to claim 4, characterized in that the nanocrystalline titanium oxide(IV) has a grain size up to 100 nm in the form of an aqueous colloidal solution.

9. The method according to claim 4, characterized in that the synthesis is carried out by the an immersion technique (for instance dip-coating technique), spraying or painting technique.

10. The method according to claim 4, characterized in that the steps take place at room temperature.

11. The method according to claim 4, characterized in that the modification of the coating surface with an organic compound takes place in aqueous or alcoholic solution of modifier with minimum concentration 10⁻⁴ mol/dm³ followed by drying.

12. The method according to claim 4, characterized in that the organic compound used for surface modification is selected from the group consisting of: phthalic acid, 4-sulfophthalic acid, 4-amino-2-hydroxybenzoic acid, 3-hydroxy-2-naphthyl acid, salicylic acid, 6-hydroxysalicylic acid, 5-hydroxysalicylic acid, 5-sulfosalicylic acid, 3,5-dinitrosalicylic acid (Table 1), 1,4-dihydroxy-1,3-benzenedisulfonic disodium salt, gallic acid, pyrogallol, 2,3-naphthalenediol, 4-methylcatechol, 3,5-di-tert-butylcatechol, p-nitrocatechol, 3,4-dihydroxy-L-phenylalanine (DOPA), catechol (Table 2), rutoside and ascorbic acid.

13. Use of photocatalytic coatings of titanium oxide(IV) according to claim 1 or obtainable in the process according to sterilization of plastic elements used in medicine, food and pharmaceuticals packaging, in particular selected from the group of: polyurethane (PU), polycarbonate (PC), polyvinyl chloride (PVC), polytetrafluoroethylene (PTFE).

14. Use of photocatalytic coatings of titanium oxide(IV) according to claim 1 or obtainable in the process according the production of selected products from the group consisting of: photosterilization materials, photobactericidal, photofungicidal, photocatalytic and other materials, especially transparent, for instance medical catheters, medical plastic tubes, foil and packaging for food and pharmaceuticals.

15. Use of photocatalytic coatings of titanium oxide(IV) according to claim 1 or obtainable in the process according to medicine particularly in dermatology, ophthalmology, otolaryngology, urology, gynecology, rheumatology, oncology, surgery, hospitality, veterinary medicine and dentistry and in cosmetology.

16. Use of photocatalytic coatings of titanium oxide(IV) according to claim 1 or obtainable in the process according to sterilization of medical catheters, plastic tubes and other surfaces, sterilization which is advantageous and/or required.
